(19) United States
(12) Patent Application Publication
(10) Pub. No.: US 2025/0275709 A1
(43) Pub. Date: Sep. 4, 2025

(54) ATRIAL FIBRILLATION INSTABILITY DIAGNOSIS AND TREATMENT

(71) Applicant: Regents of the University of Minnesota, Minneapolis, MN (US)

(72) Inventor: Alena Talkachova, Shoreview, MN (US)

(21) Appl. No.: 19/061,008

(22) Filed: Feb. 24, 2025

Related U.S. Application Data
(60) Provisional application No. 63/560,010, filed on Mar. 1, 2024.

(51) Int. Cl. A61B 5/361; A61B 5/339

(52) U.S. Cl. CPC A61B 5/361; A61B 5/339

(57) ABSTRACT

In a method of identifying a type of AF complexity and/or a treatment of AF based on the type of AF complexity, electrocardiogram (ECG) data associated with a patient is received, the ECG data includes AF signals corresponding to AF episodes. An AF burden (AFB) score is calculated using the ECG data based upon a frequency of the AF episodes or a duration of the AF episodes. An electrical burden (EB) score indicating a variation and distribution of electrical properties of the AF signals is calculated based on EB values of the ECG data in accordance with a plurality of approaches. A complexity AF score is calculated by summing the AFB and the EB scores. A type of AF complexity and/or a treatment is identified based on the complexity AF score. The complexity AF score, the identified type and/or the identified treatment is output on a display device.

### FIELD

Embodiments of the present disclosure relate to systems and methods for diagnosing particular types of Atrial Fibrillation using electrocardiogram data and identifying a targeted treatment of the diagnosed condition.

### BACKGROUND

Atrial Fibrillation (AF) is a common cardiac arrhythmia in the United States that increases the risk of stroke and can lead to other fatal heart disease. By 2050, it is estimated that 6-12 million people in the United States will have AF.

Clinically, AF is categorized into paroxysmal, persistent, long-standing persistent, and permanent AF. AF typically begins as paroxysmal episodes but can progress to more severe forms that do not resolve spontaneously. As AF progresses, the risk of cardiovascular complications and worsened symptoms increases. Early identification and appropriate management of AF progression are crucial for improving patient outcomes and treatment strategies. However, most data and analysis on AF and AF progression are gathered with intermittent and short rhythm monitoring, providing limited information on the total burden and the temporal pattern of AF.

Various metrics have been developed to quantify the complexity and severity of AF by assessing its presence in electrocardiograms (ECGs), including AF burden (AFB). Different definitions of AFB have been presented, such as the duration of the longest AF episode, and the total number of AF episodes during a monitoring period. Typically, AFB is calculated as the percentage of time that a patient is in AF, and does not account for any information regarding complexity of the AF ECG signal. Therefore, current AFB metrics have several limitations preventing them from becoming reliable clinical tools for therapeutic decisions regarding AF management, such as stroke risk and mortality. Assessing risk of AF from different aspects remains crucial for improving patient outcomes.

Using various aspects of AF signal characteristics for risk assessment helps improve patient outcomes. At present, several prognostic scores are available to identify patients at risk of developing sustained forms of AF. Many prognostic scores incorporate question-based scales for risk assessment, which determine frequency, duration, and severity of AF episodes for risk stratification. However, these scores may be challenging to use in everyday clinical practice, due to the qualitative nature of patient responses. Other risk assessments such as the HATCH score and Framingham-AF incorporate quantitative metrics only.

### SUMMARY

Embodiments of the present disclosure operate to improve the treatment of AF using a complexity AF score that is based on the intrinsic complexity of ECG signals recorded during AF. The complexity AF score is used to identify a type of AF condition or AF complexity the patient has, which may be used to identify a corresponding treatment for the AF condition.

In a computer-implemented method of identifying a type of AF complexity and/or a treatment of AF based on the type of AF complexity according to one embodiment, electrocardiogram (ECG) data associated with a patient undergoing a series of AF episodes is received, the ECG data includes AF signals corresponding to the AF episodes. An AF burden (AFB) score is calculated using the ECG data based upon a frequency of the AF episodes or a duration of the AF episodes. An electrical burden (EB) score indicating a variation and distribution of electrical properties of the AF signals is calculated based on EB values of the ECG data corresponding to a AF complexity metrics calculated in accordance with a plurality of approaches. A complexity AF score is calculated by summing the AFB and the EB scores. A type of AF complexity and/or a treatment corresponding to the type of AF complexity is identified based on the complexity AF score. The complexity AF score, the type of AF complexity, and/or the treatment for the type of AF complexity is output on a display device.

One embodiment of a system for identifying a type of atrial fibrillation (AF) complexity and/or a treatment of AF based on the type of AF includes memory, a display device and a controller. The controller is configured to: receive electrocardiogram (ECG) data associated with a patient undergoing a series of AF episodes, the ECG data including AF signals corresponding to the AF episodes; calculate an AF burden (AFB) score using the ECG data based upon a frequency of the AF episodes or a duration of the AF episodes; calculate an electrical burden (EB) score indicating a variation and distribution of electrical properties of the AF signals based on EB values of the ECG data corresponding to a AF complexity metrics calculated in accordance with a plurality of approaches; calculate a complexity AF score comprising a sum of the AFB and the EB scores; identify a type of AF complexity and/or a treatment corresponding to the type of AF complexity based on the complexity AF score; and output the complexity AF score, the type of AF complexity, and/or the treatment for the type of AF complexity on a display device.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the Background.

### BRIEF DESCRIPTION OF THE DRAWINGS

FIG. 1 is a flowchart illustrating a method of treating an AF condition of a patient, in accordance with embodiments of the present disclosure.

FIG. 2 is a simplified diagram of an example system for performing one or more steps of the method, in accordance with embodiments of the present disclosure.

FIG. 3 is a table illustrating an example of categorizations (low, middle or high) of AFB values and average EB values and their corresponding EB score.

FIG. 4 is a simplified diagram of an example of a controller, in accordance with embodiments of the present disclosure.

### DETAILED DESCRIPTION OF ILLUSTRATIVE EMBODIMENTS

Embodiments of the present disclosure are described more fully hereinafter with reference to the accompanying drawings. Elements that are identified using the same or similar reference characters refer to the same or similar elements. The various embodiments of the present disclosure may, however, be embodied in many different forms and should not be construed as limited to the embodiments set forth herein. Rather, these embodiments are provided so that this disclosure will be thorough and complete, and will fully convey the scope of the present disclosure to those skilled in the art.

The diagnosis of medical conditions determines the corresponding treatments for the conditions to minimize the effects of the conditions. As mentioned above, patients suffering from AF may fall into different categories (e.g., paroxysmal, persistent, long-standing persistent, and permanent AF) having different treatments. For example, patients with paroxysmal AF may be successfully treated with a drug regimen that may provide little benefit to a patient with sustained or permanent AF. Instead, heart tissue ablation treatments are typically more effective in treating patients with sustained or permanent AF. Therefore, it is accepted that paroxysmal AF is typically less complex than persistent and permanent AF, and therefore might require different treatment approaches.

Embodiments of the present disclosure relate to methods of identifying a complexity of a particular type of AF condition a patient has from among a plurality of different types of AF conditions, for which different treatments are recommended, based on a complexity AF score. The complexity AF score incorporates both an AFB score and an EB score. The AFB score reflects the frequency or duration of AF episodes, while the EB score assesses the variation and distribution of AF signals' electrical properties over time, using different metrics. The combination of these metrics into the complexity AF score provides a more comprehensive understanding of AF complexity and aids in the identification of high-risk patients who may benefit from more intensive monitoring or therapeutic interventions. As a result, the complexity AF score may be used to identify a complexity of a particular type of AF and a corresponding treatment to treat the AF condition. Accordingly, the methods of the present disclosure allow a physician to prescribe and/or apply a recommended treatment for a patient's AF condition, while avoiding potentially unnecessary and ineffective treatments. As a result, embodiments of the present disclosure operate to improve the efficiency and efficacy of AF treatments.

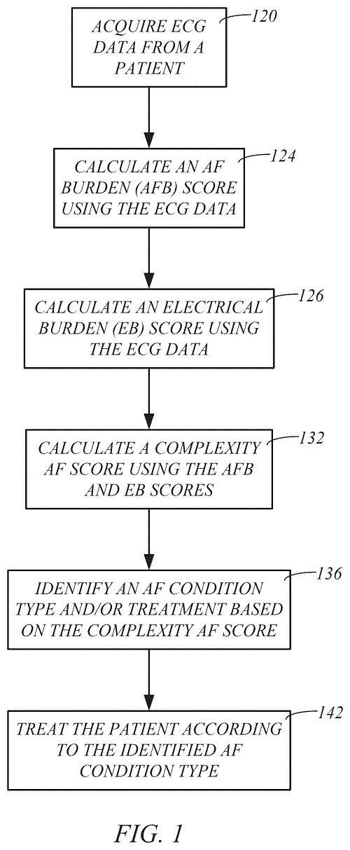

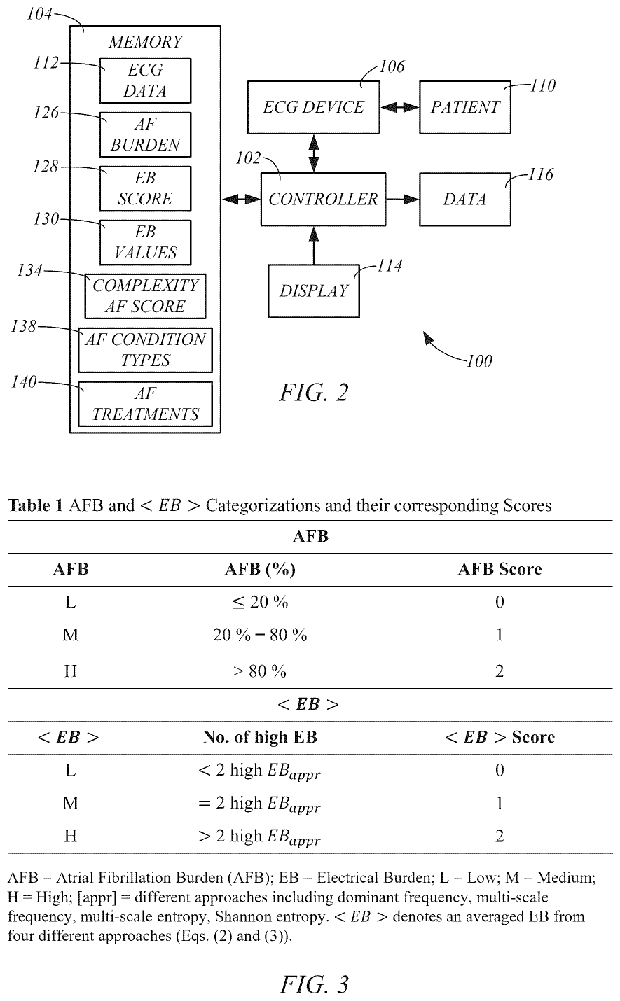

FIG. 1 is a flowchart illustrating a method of treating an AF condition of a patient, in accordance with embodiments of the present disclosure. FIG. 2 is an example of a system 100 for performing one or more steps of the method.

Some embodiments of the method utilize ECGs, which are a common cardiovascular diagnostic test used to diagnose and treat different heart conditions including AF. ECGs generally relate to a measurement of electrical potential differentials or voltage differences between various locations on the surface of a patient's body. The ECG is formed by a plot of the voltage difference over time, such as at a rate of 300-500 voltage difference samples per second.

Embodiments of the system 100 include a controller 102, memory 104, an ECG device 106, and/or a display 114. The memory 104 includes one or more memory devices or databases, and represents local memory, remote memory, and/or memory of the controller 102, for example. The controller 102 generally operates to perform one or more functions described herein including steps of the method of FIG. 1, in response to the execution of program instructions, which may be stored in the memory 104, memory of the controller 102, and/or another source of memory. The memory 104 and other memory described herein includes any suitable patent eligible memory and does not include transitory waves or signals.

The ECG device 106 may be a conventional device used to collect ECG data of a patient 110. ECG data 112 that is acquired using the ECG device 106 may be stored in the memory 104 for use by the controller 102.

The controller 102 processes the ECG data 112 that is received from the ECG device 106 for a patient and/or obtained from the memory 104 to identify a type of AF complexity and/or AF condition the patient has, and the corresponding treatment, in accordance with the method of FIG. 1. The system 100 may include a display 114 for displaying information, such as the identified type of AF condition and/or the corresponding treatment for the AF condition. The controller 102 may communicate data 116 using conventional techniques, such as data relating to the ECG data 112, the identified AF condition type, the determined treatment, and/or other information, such as the calculated values discussed below and information stored in the memory 104, for example.

At 120 of the method, ECG data 112 is acquired from a patient 110 during an episode of AF. This ECG data 112 may be collected and obtained using the ECG device 106 and/or obtained from the memory 104. The ECG data 112 may comprise distinct electrical potential measurements taken over a period of time in accordance with conventional techniques. For example, the ECG data 112 may be collected at a frequency of about 128 Hz. Additionally, the ECG device 106 or the controller 102 may filter the collected ECG data 112, such as using a bandpass filter (e.g., 0.5-40 Hz) to remove baseline wander and/or noise, for example. Also, the ECG data 112 may relate to a single examination of the patient 110 or multiple examinations of the patient 110.

At 124 of the method, the controller 102 calculates an AFB score 126, which indicates a frequency or duration of AF episodes in the ECG data. In one embodiment, the ECG data 112 is processed by the controller 102 to identify the duration of any AF episodes and the total recording duration. The AF episodes may be identified by the controller using conventional techniques, such as change in heart rate variability, and the duration may be determined based on a sampling frequency of the ECG data or a corresponding time measurement, or through a manual process.

In one embodiment, an AFB value for the ECG data 112 is determined by calculating the percentage of time that the patient has AF rhythm as defined by Equation 1.

$$AFB = \frac{total duration of AF rhythm}{total duration of recording} \times 100. \qquad ( 1 )$$

In some embodiments, the AFB value may be classified as low (L), medium (M), or high (H). The classification of the AFB values may be based on predefined thresholds. For example, a threshold AFB value of 20% may separate the low classification from medium classification, and a threshold AFB value of 80% may separate the medium classification from the high classification, as indicated in Table 1 of FIG. 3.

In one embodiment, the AFB score is assigned based on the classification of the AFB value. For example, an AFB score of 0 may be assigned when the AFB value is classified as low, an AFB score of 1 may be assigned when the AFB value is classified as medium, and an AFB score of 2 may be assigned when the AFB value is classified as high, as indicated in FIG. 3. In one example, an AFB value less than about 20% may be classified as low, an AFB value in the range of about 20-80% may be classified as medium, and an AFB value greater than about 80% may be classified as high.

At 126 of the method, an EB score 128 is calculated or determined using the controller 102 and is used to assess electrical instability in AF signals. Studies have shown that the EB does not correlate with AFB and can have different values for both low AFB as well as for high AFB. Accordingly, the electrical instability of the AF signal has been found to be an independent and supplemental marker to the AFB score, thus describing another aspect of complexity of AF signals.

Additionally, temporal changes in complexity of dominant frequency (DF), multi-scale frequency (MSF), multi-scale entropy (MSE), and Shannon entropy (SE) do not directly correlate with the AFB. For instance, DF and SE complexity over time may be different between patients with high AFB and patients with low AFB. These findings underscore the distinction between EB and AFB, highlighting their divergent roles in characterizing ECG signals.

Step 126 may be performed before, after, concurrently or partially concurrently with the performance of step 124. In one embodiment, the controller 102 calculates or determines the EB score 128 based on EB values 130. The EB values 130 may be calculated by partitioning the ECG data 112 into predefined segments, each corresponding to a time interval. Embodiments of the time intervals include include 10-60 second intervals, such as 30-second intervals, or other time intervals. Thus, when the ECG data 112 comprises data from a 20 hour ECG recording, the ECG data 112 may be divided into 2400 30-second segments.

EB values corresponding to conventional AF complexity metrics may be calculated for each ECG data segment using the controller 102 in accordance with various approaches including DF, MSF, MSE, SE, and/or other approaches. The controller 102 may then determine a percentage of signals that deviate beyond one standard deviation (SD) from the distribution of AF complexity, measured through the different approaches ([appr]). For example, the controller 102 may perform a calculation for EB values 130 as indicated in Equation 2.

$$E {B}_{[ appr ]} = \frac{count ( \vert [ appr ] values - \\ mean ( [ appr ] values ) \vert > SD ( [ appr ] values ) )}{N ( [ appr ] values )} \times 100 . \qquad ( 2 )$$

Here, the notation N([appr] values) denotes the total number of estimated values for each approach (DF, MSF, MSE, SE etc), which relates to the number of ECG data segments that are being analyzed. For example, when the ECG data 112 is divided into 2400 30-second segments from a 20 hour ECG recording, 2400 ECG data segments are analyzed by the controller 102 under each approach.

In some embodiments, a number of outliers in the AF complexity metrics (numerator), EB_{[appr]} represent different levels of ECG instability, based on the specific approach used to calculate it. Therefore, it can reflect, for example, frequency-based, statistical-based, and information-based complexity of the ECG data 112 (e.g., signal or recording) during AF.

Each of the EB values 130 calculated using Eq. 2 may be classified in step 126 as a low complexity value indicating electrical stability of the ECG signal or data 112, or a high complexity value indicating electrical instability in the ECG signal or data 112 based on a threshold EB value. The threshold EB value for designating the EB values as low or high complexity may be set based on the particular approach. For example, an EB value threshold of 25% may be used, such that an EB value 130 of less than or equal to 25% is classified as low complexity and an EB value of greater than 25% is classified as high complexity.

To ensure an equal contribution from the EB and AFB calculations, the average of the EB values (<EB>) calculated for the different approaches may be classified as low (L), medium (M), or high (H) based on a number of EB values (e.g., 2) classified as high complexity, and the EB score is assigned based on the classification, as indicated in Table 1 of FIG. 3. For example, an EB score of 0 may be assigned when the average EB value is classified as low, an EB score of 1 may be assigned when the average EB value is classified as medium, and an EB score of 2 may be assigned when the average EB value is classified as high, as indicated in FIG. 3.

At 132 of the method, a complexity AF score 134 for a patient is calculated or determined using the controller 102 based on the AFB score 126 and the EB score 128 for the patient. The complexity AF score 134 indicates a likelihood of a patient developing complex AF. The incorporation of both the AFB and EB in the complexity AF score provides valuable insights into the temporal and electrical instability of the AF signals in the ECG data. The AFB score 126 reflects the frequency or duration of AF episodes, while the EB score 128 assesses the variation and distribution of AF signals' electrical properties over time, using different metrics or approaches. Combining these metrics provides a more comprehensive understanding of AF complexity, aiding in the identification of high-risk patients who may benefit from more intensive monitoring or therapeutic interventions. Thus, the complexity AF score is effective at describing the complexity of AF, such as paroxysmal AF and persistent AF and provides a more comprehensive evaluation of AF dynamics than that provided through an analysis of the AFB score 126 and the EB score 128 individually. As a result, the complexity AF score 134 may be used to determine effective targeted treatment strategies for AF patients, while avoiding potentially unnecessary and ineffective treatments.

Patients with a low AFB score 126 and a low EB score 128 have a lower risk of developing complex AF. This is because both time-based complexity metrics of the AF signals are classified as low. Conversely, patients with a high AFB score 126 and a high EB score 128 have a higher risk of developing complex AF due to unstable ECG signals.

In one embodiment, the complexity AF score 134 is calculated in step 132 by summing the AFB score 126 and the EB score 128. Thus, the AFB score 126 and the EB score 128 may be assigned values based on a complexity level as described above with reference to Table 1, and the complexity AF score 134 may be calculated by adding the scores corresponding to the complexity levels together. Based on these example AFB scores 126 and EB scores 128, the complexity AF score can range from 0-4. A complexity AF score 134 of zero (0) may, for example, indicate sinus rhythm, a low complexity AF score of 1-2 may indicate that the patient has a moderate chance of developing complex AF, and a high AF score of 3-4 may indicate that the patient has a high chance of developing complex AF.

The complexity AF score 134 may be stored in the memory 104. In some embodiments, the controller 102 may output information that includes or relates to the AF score, such as by communicating the information to a database, issuing a report (e.g., document, email, etc.) containing the information, and/or outputting the information in another form, for example.

At 136 of the method, a type of AF condition 138 and/or a corresponding recommended treatment 140 may be identified by the controller 102, such as through a mapping of complexity AF scores 134 to a plurality of AF condition types 138 and treatments 140 stored in the memory 104.

Examples of the AF condition types that may be identified based on a complexity AF score include paroxysmal and persistent AF. Examples of the recommended treatments 140 that correspond to the identified condition types include a drug regimen, such as heart rate controlling drugs (for example, beta blockers and calcium channel blockers), or heart rhythm controlling drugs (for example, potassium and sodium channel blockers), for low complexity AF scores 134, and an ablation treatment of heart tissue may be recommended for high complexity AF scores 134.

Information that includes or relates to the AF condition type 138 and/or the recommend treatment 140 may be stored in the memory 104 and/or output by the controller 102, such as by communicating the information to a database, issuing a report (e.g., document, email, etc.) containing the information, and/or outputting the information in another form, for example.

In some embodiments, the AF condition type 138 of the patient may be treated at 142 of the method using the recommended treatment 140 corresponding to the complexity AF score 134 or AF condition type 138.

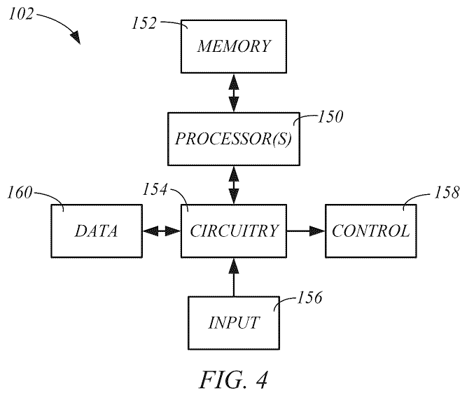

The controller 102 may take on any suitable form and may represent a single controller or multiple controllers. FIG. 4 is a simplified diagram of an example of the controller 102 in accordance with embodiments of the present disclosure. The controller 102 may include one or more processors 150 and memory 152. The one or more processors 150 are configured to perform various functions of the system 100 described herein, such as steps of the method of FIG. 1, and/or other functions described herein, in response to the execution of instructions contained in the memory 152.

The one or more processors 150 may be components of one or more computer-based systems, and may include one or more control circuits, microprocessor-based engine control systems, and/or one or more programmable hardware components, such as a field programmable gate array (FPGA). The memory 152 represents local and/or remote memory or computer readable media and may represent the memory 104 of the system 100. Such memory 152 comprises any suitable patent subject matter eligible computer readable media and does not include transitory waves or signals. Examples of the memory 152 include conventional data storage devices, such as hard disks, CD-ROMs, optical storage devices, magnetic storage devices and/or other suitable data storage devices. The controller 102 may include circuitry 154 for use by the one or more processors 150 to receive input signals 156 (e.g., ECG signals), issue control signals 158 (e.g., signals that control the display 114, etc.), and/or communicate data 160, such as in response to the execution of the instructions stored in the memory 152 by the one or more processors 150.

Although the embodiments of the present disclosure have been described with reference to preferred embodiments. workers skilled in the art will recognize that changes may be made in form and detail without departing from the spirit and scope of the present disclosure.

What is claimed is:

1. A computer-implemented method of identifying a type of atrial fibrillation (AF) complexity and/or a treatment of AF based on the type of AF comprising:
receiving electrocardiogram (ECG) data associated with a patient undergoing a series of AF episodes, the ECG data including AF signals corresponding to the AF episodes;
calculating an AF burden (AFB) score using the ECG data based upon a frequency of the AF episodes or a duration of the AF episodes;
calculating an electrical burden (EB) score indicating a variation and distribution of electrical properties of the AF signals based on EB values of the ECG data corresponding to a AF complexity metrics calculated in accordance with a plurality of approaches;
calculating a complexity AF score comprising a sum of the AFB and the EB scores;
identifying a type of AF complexity and/or a treatment corresponding to the type of AF complexity based on the complexity AF score; and
outputting the complexity AF score, the type of AF complexity, and/or the treatment for the type of AF complexity on a display device.

2. The method according to claim 1 wherein:
identifying the type of AF complexity and/or the treatment corresponding to the type of AF complexity based on the complexity AF score comprises identifying the type of AF and the treatment corresponding to the identified type of AF based on the complexity AF score;
outputting the complexity AF score, the type of AF complexity and/or the treatment for the type of AF complexity comprises outputting the AF score, the type of AF complexity and the treatment on the display device; and
the method includes treating the patient in accordance with the identified treatment.

3. The method according to claim 2, wherein the treatment is selected from the group consisting of:
applying a drug regimen to the patient when the AF score indicates a low complexity type of AF; and
ablating heart tissue of the patient when the AF score indicates a high complexity type of AF.

4. The method according to claim 1, wherein the AFB score is calculated based on an AFB value, which is calculated using the following equation:
$$AFB = \frac{total duration of AF rhythm}{total duration of recording} \times 100 ,$$
wherein the “total duration of AF rhythm” corresponds to a duration of the AF episodes, and the “total duration of recording” corresponds to the period corresponding to the ECG data.

5. The method according to claim 4, wherein the EB values of the ECG data for each approach (EB[appr]) are calculated based on the following equation:
$$E {B}_{[ appr ]} = \frac{count ( \vert [ appr ] values - \\ mean ( [ appr ] values ) \vert > SD ( [ appr ] values ) )}{N ( [ appr ] values )} \times 100 ,$$
wherein “appr” corresponds to an AF complexity metric calculated using the approach, “[appr] values” denotes AF complexity metric values of the AF signals in the ECG data calculated using the particular approach, “count(|[appr]values−mean([appr] values|)>SD([appr] values” denotes a count of the absolute value of the [appr] values minus the mean of the [appr] values being greater than a standard deviation of the [appr] values, and N([appr] values) denotes a total number of the [appr] values.

6. The method according to claim 5, wherein the ECG data is partitioned into predefined segments, and each [appr] value corresponds to the AF complexity metric value in one of the segments.

7. The method according to claim 6, wherein the predefined segments of the ECG data each correspond to a time interval of 10-60 seconds.

8. The method according to claim 6, wherein the plurality of approaches are selected from the group consisting of dominant frequency (DF), multiscale frequency (MSF), multiscale entropy (MSE) and Shannon entropy (SE).

9. The method according to claim 6, wherein the EB score is based on an average of the EB values from the plurality of approaches.

10. The method according to claim 9, including:
classifying the average of the EB values as low complexity indicating electrical stability or high complexity indicating electrical instability based on a threshold EB value; and
assigning the EB score based on a number of the EB values classified as high complexity.

11. The method according to claim 10, including:
classifying the AFB value as low complexity, a medium complexity or a high complexity; and
assigning the AFB score based on the classification of the AFB value.

12. A system for identifying a type of atrial fibrillation (AF) complexity and/or a treatment of AF based on the type of AF comprises:
memory;
a display device; and
a controller configured to:
receive electrocardiogram (ECG) data associated with a patient undergoing a series of AF episodes, the ECG data including AF signals corresponding to the AF episodes;
calculate an AF burden (AFB) score using the ECG data based upon a frequency of the AF episodes or a duration of the AF episodes;
calculate an electrical burden (EB) score indicating a variation and distribution of electrical properties of the AF signals based on EB values of the ECG data corresponding to a AF complexity metrics calculated in accordance with a plurality of approaches;
calculate a complexity AF score comprising a sum of the AFB and the EB scores;
identify a type of AF complexity and/or a treatment corresponding to the type of AF complexity based on the complexity AF score; and
output the complexity AF score, the type of AF complexity, and/or the treatment for the type of AF complexity on a display device.

13. The system according to claim 12 wherein:
the identification of the type of AF complexity and/or the treatment corresponding to the type of AF complexity identified based on the complexity AF score comprises identifying the type of AF and the treatment corresponding to the identified type of AF based on the complexity AF score; and
the output of the complexity AF score, the type of AF complexity and/or the treatment for the type of AF complexity comprises an output of the AF score, the type of AF complexity and the treatment on the display device.

14. The system according to claim 13, wherein the identified treatment is selected from the group consisting of:
applying a drug regimen to the patient when the AF score indicates a low complexity type of AF; and
ablating heart tissue of the patient when the AF score indicates a high complexity type of AF.

15. The system according to claim 12, wherein the AFB score is calculated based on an AFB value, which is calculated using the following equation:
$$AFB = \frac{total duration of AF rhythm}{total duration of recording} \times 100 ,$$
wherein the “total duration of AF rhythm” corresponds to a duration of the AF episodes, and the “total duration of recording” corresponds to the period corresponding to the ECG data.

16. The system according to claim 15, wherein the EB values of the ECG data for each approach (EB[appr]) are calculated based on the following equation:
$$E {B}_{[ appr ]} = \frac{count ( \vert [ appr ] values - \\ mean ( [ appr ] values ) \vert > SD ( [ appr ] values ) )}{N ( [ appr ] values )} \times 100 ,$$
wherein “appr” corresponds to an AF complexity metric calculated using the approach, “[appr] values” denotes AF complexity metric values of the AF signals in the ECG data calculated using the particular approach, “count(|[appr]values−mean([appr] values|)>SD([appr] values” denotes a count of the absolute value of the [appr] values minus the mean of the [appr] values being greater than a standard deviation of the [appr] values, and N([appr] values) denotes a total number of the [appr] values.

17. The system according to claim 16, wherein the controller is configured to partition the ECG data into predefined segments, and each [appr] value corresponds to the AF complexity metric value in one of the segments.

18. The system according to claim 17, wherein the plurality of approaches are selected from the group consisting of dominant frequency (DF), multiscale frequency (MSF), multiscale entropy (MSE) and Shannon entropy (SE).

19. The system according to claim 18, wherein the controller is configured to:
classify an average of the EB values as low complexity indicating electrical stability or high complexity indicating electrical instability based on a threshold EB value; and
assigning the EB score based on a number of the EB values classified as high complexity.

20. The system according to claim 19, wherein the controller is configured to:
classify the AFB value as low complexity, a medium complexity or a high complexity; and
assign the AFB score based on the classification of the AFB value.
